(19) United States
(12) Patent Application Publication
(10) Pub. No.: US 2011/0229548 A1
(43) Pub. Date: Sep. 22, 2011

(54) Implantable Medical Device With Poly(Vinylidene Fluoride-Co-Hexafluoropropylene)/Zotarolimus Drug Layer

(75) Inventors: Florencia Lim, Union City, CA (US); Shawn Chin Quee, Santa Clara, CA (US); Mikael O. Trollsas, San Jose, CA (US)

(73) Assignee: Abbott Cardiovascular Systems Inc., Santa Clara, CA (US)

(21) Appl. No.: 12/727,175

(22) Filed: Mar. 18, 2010

(51) Int. Cl. A61F 2/82; A61K 31/436; A61F 2/06

(52) U.S. Cl. 424/423; 514/291; 623/1.42

(57) ABSTRACT

This invention relates to an implantable medical device for and method of treating a vascular disease where the device is preferably a stent comprising a drug reservoir layer comprising poly(vinylidene fluoride-co-hexafluoropropylene) and zotarolimus.

### FIELD

This invention relates to the field of medical devices and methods of their use, in particular to drug-eluting stents.

### BACKGROUND

The discussion that follows is intended solely as background information to assist in the understanding of this invention; nothing in this section is intended to be, nor is it to be construed as, prior art to this invention.

Until the mid-1980s, the accepted treatment for atherosclerosis, i.e., narrowing of the coronary artery(ies) was coronary by-pass surgery. While effective and while having evolved to a relatively high degree of safety for such an invasive procedure, by-pass surgery still entails potentially serious complications and, in the best of cases, an extended recovery period.

With the advent of percutaneous transluminal coronary angioplasty (PTCA) in 1977, the scene changed dramatically. Using catheter techniques originally developed for heart exploration, inflatable balloons were employed to re-open occluded regions in arteries. The procedure was relatively non-invasive, took a very short time compared to by-pass surgery and the recovery time was minimal. However, PTCA brought with it another problem, elastic recoil of the stretched arterial wall which could undo much of what had been accomplished and, in addition, PTCA failed to satisfactorily ameliorate another problem, restenosis, the re-clogging of the treated artery.

The next improvement, advanced in the mid-1980s, was use of a stent to scaffold the vessel wall in place after PTCA. This for all intents and purposes put an end to elastic recoil but did not entirely resolve the issue of restenosis. That is, prior to the introduction of stents, restenosis occurred in from 30-50% of patients undergoing PTCA. Stenting reduced this to about 15-20%, much improved but still more than desirable.

In 2003, drug-eluting stents or DESs were introduced. The drugs initially employed with DESs were cytostatic compounds, compounds that curtailed the proliferation of cells that engendered restenosis. The occurrence of restenosis was thus reduced to about 5-7%, a relatively acceptable figure. Today, the DES is the default industry standard for the treatment of atherosclerosis and is rapidly gaining favor for treatment of stenoses of blood vessels other than coronary arteries such as peripheral angioplasty of the superficial femoral artery.

There continues to be a need for more novel DESs to more effectively reduce the occurrence of, preferably to eliminate, restenosis in stented patients. The present invention provides such a novel DES and methods of its use.

### SUMMARY

Thus, in one aspect, the current invention relates to an implantable medical device comprising:

a device body;
an optional primer layer disposed over at least a portion of the device body;
a drug reservoir layer disposed over at least a portion of the device body or, if opted for, over the primer layer, wherein

the drug reservoir layer comprises poly(vinylidene fluoride-co-hexafluoropropylene), zotarolimdus, optionally one or more additional therapeutic agents; and

an optional topcoat layer.

In an aspect of this invention, the weight ratio of zotarolimus to poly(vinylidene fluoride-co-hexafluoropropylene) is about 1:1 to about 1:10.

In an aspect of this invention, the weight ratio of zotarolimus to poly(vinylidene fluoride-co-hexafluoropropylene) is about 1:3 to about 1:5.

In an aspect of this invention, the weight ratio of zotarolimus to poly(vinylidene fluoride-co-hexafluoropropylene) is about 1:3.

In an aspect of this invention, zotarolimus is present in the drug delivery layer at about 10 μg/cm² to about 1000 μg/cm².

In an aspect of this invention, zotarolimus is present in the drug delivery layer at about 50 μg/cm² to about 500 μg/cm².

In an aspect of this invention, zotarolimus is present in the drug delivery layer at about 100 μg/cm².

In an aspect of this invention, approximately 23% of the zotarolimus elutes from the drug reservoir layer into the surrounding environment after about 24 hours.

In an aspect of this invention, approximately 40% of the zotarolimus elutes from the drug reservoir layer into the surrounding environment after about 40 hours.

In an aspect of this invention, the device is sterile.

In an aspect of this invention, the device is sterilized by exposure to ethylene oxide.

In an aspect of this invention the topcoat layer is not opted for.

In an aspect of this invention, the primer layer is not opted for.

In an aspect of this invention, neither the topcoat layer nor the primer layer is opted for.

In an aspect of this invention, the device body comprises a stent.

An aspect of this invention is related to a method comprising:

identifying a patient afflicted with a vascular disease;
inserting an implantable device percutaneously into a vessel of the patient at a site of the vascular disease, wherein the implantable medical device comprises:
a device body;
an optional primer layer disposed over at least a portion of the device body;
a drug reservoir layer disposed over at least a portion of the device body or, if opted for, over the primer layer, wherein
   the drug reservoir layer comprises poly(vinylidene fluoride-co-hexafluoropropylene), zotarolimdus, optionally one or more additional therapeutic agents; and
an optional topcoat layer.

In an aspect of this invention, in the above method the topcoat layer is not opted for.

In an aspect of this invention, in the above method the primer layer is not opted for.

In an aspect of this invention, in the above method no additional therapeutic agent is opted for.

In an aspect of this invention, in the above method the device body comprises a stent.

In an aspect of this invention, in the above method the vascular disease is selected from the group consisting of atherosclerosis, restenosis, vulnerable plaque and superficial femoral artery sclerosis.

### DETAILED DESCRIPTION

It is understood that use of the singular throughout this application including the claims includes the plural and vice versa unless expressly stated otherwise. That is, “a” and “the” are to be construed as referring to one or more of whatever the word modifies. Non-limiting examples are: “a therapeutic agent,” which is understood to include one such agent, two such agents or, under the right circumstances, as determined by those skilled in the treatment of diseased tissues, even more such agents unless it is expressly stated or is unambiguously obvious from the context that such is not intended. Likewise, “a biodegradable polymer” refers to a single polymer or a mixture of two or more polymers unless, again, it is expressly stated or absolutely obvious from the context that such is not intended.

As used herein, words of approximation such as, without limitation, “about” “substantially,” “essentially” and “approximately” mean that the word or phrase modified by the term need not be exactly that which is written but may vary from that written description to some extent. The extent to which the description may vary will depend on how great a change can be instituted and have one of ordinary skill in the art recognize the modified version as still having the properties, characteristics and capabilities of the modified word or phrase. In general, but modifiable by the preceding discussion, a numerical value herein that is modified by a word of approximation may vary from the stated value by ±15%.

As used herein, the use of “preferred,” “preferably,” or “more preferred,” and the like refer to modify an aspect of the invention refers to preferences as they existed at the time of filing of the patent application.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves and cerebrospinal fluid shunts.

As used herein, “device body” refers to a fully formed implantable medical with an outer surface to which no coating or layer of material different from that of which the device itself is fabricated has been applied. By “outer surface” is meant any surface however spatially oriented that is in contact with bodily tissue or fluids. A common example of a “device body” is a BMS, i.e., a bare metal stent, which, as the name implies, is a fully-formed usable stent that has not been coated with a layer of any material different from the metal of which it is made on any surface that is in contact with bodily tissue or fluids. Of course, device body refers not only to BMSs but to any uncoated device regardless of what it is made of.

At present, preferred implantable medical devices for use with the coatings of this invention are stents.

A stent refers generally to any device used to hold tissue in place in a patient's body. Particularly useful stents, however, are those used for the maintenance of the patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, restenosis and vulnerable plaque. Vulnerable plaque (VP) refers to a fatty build-up in an artery thought to be caused by inflammation. The VP is covered by a thin fibrous cap that can rupture leading to blood clot formation. A stent can be used to strengthen the wall of the vessel in the vicinity of the VP and act as a shield against such rupture. A stent can be used in, without limitation, neuro, carotid, coronary, pulmonary, aorta, renal, biliary, iliac, femoral and popliteal as well as other peripheral vasculatures. A stent can be used in the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

In addition to the above uses, stents may also be employed for the localized delivery of therapeutic agents to specific treatment sites in a patient's body. In fact, therapeutic agent delivery may be the sole purpose of the stent or the stent may be primarily intended for another use such as those discussed above with drug delivery providing an ancillary benefit.

A stent used for patency maintenance is usually delivered to the target site in a compressed state and then expanded to fit the vessel into which it has been inserted. Once at a target location, a stent may be self-expandable or balloon expandable.

As used herein, “optional” means that the element modified by the term may, but is not required to, be present.

As used herein, a “primer layer” refers to a coating consisting of a polymer or blend of polymers that exhibit good adhesion characteristics with regard to the material of which the device body is fabricated and good adhesion characteristics with regard to whatever material is to be applied to a surface of the device body. Thus, a primer layer serves as an intermediary layer between a device body and materials to be affixed to the device body and is, therefore, applied directly to the device body. Examples without limitation, of primers include acrylate and methacrylate polymers with poly(n-butyl methacrylate) being a presently preferred primer. Some additional examples of primers include, but are not limited to, poly(ethylene-co-vinyl alcohol), poly(vinyl acetate-co-vinyl alcohol), poly(methacrylates), poly(acrylates), polyethyleneamine, polyallylamine, chitosan, poly(ethylene-co-vinyl acetate), and parylene-C.

As use herein, a material that is described as a layer “disposed over” an indicated substrate, e.g., without limitation, a device body or another layer, refers to a relatively thin coating of the material applied, preferably at present, directly to essentially the entire exposed surface of the indicated substrate. By “exposed surface” is meant any surface regardless of its physical location with respect to the configuration of the device that, in use, would be in contact with bodily tissues or fluids. “Disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, “drug reservoir layer” refers either to a layer of one or more therapeutic agents applied neat or to a layer of polymer or blend of polymers that has dispersed within its three-dimensional structure one or more therapeutic agents. A polymeric drug reservoir layer is designed such that, by one mechanism or another, e.g., without limitation, by elution or as the result of biodegradation of the polymer, the therapeutic substance is released from the layer into the surrounding environment. For the purpose of this invention, the drug reservoir layer also acts as rate-controlling layer. As used herein, “rate-controlling layer” refers to a polymer layer that controls the rate of release of therapeutic agents or drugs into the environment. While any polymer may be used to construct a drug reservoir layer of this invention, particularly useful polymer includes, but not limited to, poly(vinylidene fluoride-co-hexafluoropropylene) (PVDF-HFP).

As used herein, a “topcoat layer” refers to an outermost layer, that is, a layer that is in contact with the external environment and that is coated over all or substantially all other layers. The topcoat layer may be applied to provide, or provide additional, control of the rate of release of the therapeutic agent from the underlying reservoir layer, to confer better hydrophilicity on the device, to make the device more biocompatible, to better lubricate the device or merely as a physical protectant of the underlying layers. The topcoat layer, however, may also contain therapeutic agents, in particular if the treatment protocol being employed calls for essentially immediate release of one or more therapeutic agent (these being included in the topcoat layer) followed by the controlled release of another therapeutic agent or agents over a longer period of time.

As used herein, “therapeutic agent” refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. As used herein, a therapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a reduced level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

As used herein, the terms “drug” and “therapeutic agent” are used interchangeably.

As used herein, “treating” refers to the administration of a therapeutically effective amount of a therapeutic agent to a patient known or suspected to be afflicted with a vascular disease.

By “afflicted with” is meant that the patient exhibits symptoms that one skilled in the medical arts would associate with and diagnose as a vascular disease.

By “identifying” a patient afflicted with a vascular disease simply means to diagnose the disease in the patient using any of the tools available to the medical profession for such diagnosis as such now exist or as may be developed in the future.

As used herein, a “patient” refers to any living organism that might benefit from the application of the implantable medical device and method of this invention. Preferable the patient is a mammal and most preferably at present the patient is a human being.

A “therapeutically effective amount” refers to that amount of a therapeutic agent that will have a beneficial affect, which may be curative or palliative, on the health and well-being of the patient with regard to the vascular disease with which the patient is known or suspected to be afflicted. A therapeutically effective amount may be administered as a single bolus, as intermittent bolus charges, as short, medium or long term sustained release formulations or as any combination of these. As used herein, short-term sustained release refers to the administration of a therapeutically effective amount of a therapeutic agent over a period from about several hours to about 3 days. Medium-term sustained release refers to administration of a therapeutically effective amount of a therapeutic agent over a period from about 3 day to about 14 days and long-term refers to the delivery of a therapeutically effective amount over any period in excess of about 14 days.

As used herein, a “vascular disease” refers to a disease of the vessels, primarily arteries and veins, which transport blood to and from the heart, brain and peripheral organs such as, without limitation, the arms, legs, kidneys and liver. In particular “vascular disease” refers to the coronary arterial system, the carotid arterial system and the peripheral arterial system. The disease that may be treated is any that is amenable to treatment with a therapeutic agent, either as the sole treatment protocol or as an adjunct to other procedures such as surgical intervention. The disease may be, without limitation, atherosclerosis, vulnerable plaque, restenosis or peripheral arterial disease.

“Atherosclerosis” refers to the depositing of fatty substances, cholesterol, cellular waste products, calcium and fibrin on the inner lining or intima of an artery. Smooth muscle cell proliferation and lipid accumulation accompany the deposition process. In addition, inflammatory substances that tend to migrate to atherosclerotic regions of an artery are thought to exacerbate the condition. The result of the accumulation of substances on the intima is the formation of fibrous (atheromatous) plaques that occlude the lumen of the artery, a process called stenosis. When the stenosis becomes severe enough, the blood supply to the organ supplied by the particular artery is depleted resulting is strokes, if the afflicted artery is a carotid artery, heart attack if the artery is a coronary artery, or loss of organ function if the artery is peripheral.

“Restenosis” refers to the re-narrowing or blockage of an artery at or near the site where angioplasty or another surgical procedure was previously performed to remove a stenosis. It is generally due to smooth muscle cell proliferation and, at times, is accompanied by thrombosis. Prior to the advent of implantable stents to maintain the patency of vessels opened by angioplasty, restenosis occurred in 40-50% of patients within 3 to 6 months of undergoing the procedure. Post-angioplasty restenosis before stents was due primarily to smooth muscle cell proliferation. There were also issues of acute re-closure due to vasospasm, dissection, and thrombosis at the site of the procedure. Stents eliminated acute closure from vasospasm and greatly reduced complications from dissections. The use of IIb-IIIa anti-platelet drugs such as abciximab and epifabatide, which are anti-thrombotic, reduced the occurrence of post-procedure clotting (although stent placement itself can initiate thrombosis). Stent placement sites are also susceptible to restenosis due to abnormal tissue growth at the site of implantation. This form of restenosis tends also to occur at 3 to 6 months after stent placement but it is not affected by the use of anti-clotting drugs. Thus, alternative therapies are continuously being sought to mitigate, preferably eliminate, this type of restenosis. Drug eluting stents (DES), such as those of the instant invention, which release a variety of therapeutic agents at the site of stent placement, have been in use for some time and, as is the case with the invention herein, are being constantly improved.

“Vulnerable plaque” refers to an atheromatous plaque that has the potential of causing a thrombotic event and is usually characterized by a very thin wall separating it from the lumen of an artery. The thinness of the wall renders the plaque susceptible to rupture. When the plaque ruptures, the inner core of usually lipid-rich plaque is exposed to blood, with the potential of causing a potentially fatal thrombotic event through adhesion and activation of platelets and plasma proteins to components of the exposed plaque.

The phenomenon of “vulnerable plaque” has created new challenges in recent years for the treatment of heart disease. Unlike occlusive plaques that impede blood flow, vulnerable plaque develops within the arterial walls, but it often does so without the characteristic substantial narrowing of the arterial lumen which produces symptoms. As such, conventional methods for detecting heart disease, such as an angiogram, may not detect vulnerable plaque growth into the arterial wall.

The intrinsic histological features that may characterize a vulnerable plaque include increased lipid content, increased macrophage, foam cell and T lymphocyte content, and reduced collagen and smooth muscle cell (SMC) content. This fibroatheroma type of vulnerable plaque is often referred to as “soft,” having a large lipid pool of lipoproteins surrounded by a fibrous cap. The fibrous cap contains mostly collagen, the reduced concentration of which, combined with macrophage-derived enzyme degradation, can cause the fibrous cap of these lesions to rupture under unpredictable circumstances. When ruptured, the lipid core contents, thought to include tissue factor, contact the arterial bloodstream, causing a blood clot to form that can completely block the artery resulting in an acute coronary syndrome (ACS) event. This type of atherosclerosis is coined “vulnerable” because of unpredictable tendency of the plaque to rupture. It is thought that hemodynamic and cardiac forces, which yield circumferential stress, shear stress, and flexion stress, may cause disruption of a fibroatheroma type of vulnerable plaque. These forces may rise as the result of simple movements, such as getting out of bed in the morning, in addition to in vivo forces related to blood flow and the beating of the heart. It is thought that plaque vulnerability in fibroatheroma types is determined primarily by factors which include: (1) size and consistency of the lipid core; (2) thickness of the fibrous cap covering the lipid core; and (3) inflammation and repair within the fibrous cap.

“Thrombosis” refers to the formation or presence of a blood clot (thrombus) inside a blood vessel or chamber of the heart. A blood clot that breaks off and travels to another part of the body is called an embolus. If a clot blocks a blood vessel that supplied blood to the heart, it causes a heart attack. If a clot blocks a blood vessel that supplied blood to the brain, it causes a stroke.

Peripheral vascular diseases are generally caused by structural changes in blood vessels caused by such conditions as inflammation and tissue damage. A subset of peripheral vascular disease is peripheral artery disease (PAD). PAD is a condition that is similar to carotid and coronary artery disease in that it is caused by the buildup of fatty deposits on the lining or intima of the artery walls. Just as blockage of the carotid artery restricts blood flow to the brain and blockage of the coronary artery restricts blood flow to the heart, blockage of the peripheral arteries can lead to restricted blood flow to the kidneys, stomach, arms, legs and feet. In particular at present a peripheral vascular disease refers to a vascular disease of the superficial femoral artery.

The presently preferred therapeutic agent for use with the device and method of this invention is zotarolimus. Zotarolimus has the chemical structure:

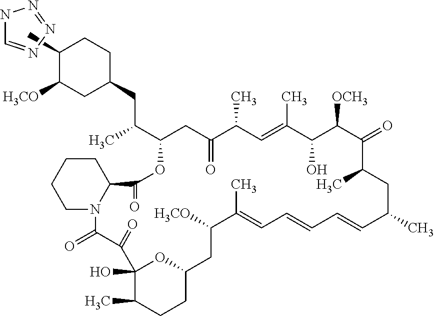

Zotarolimus is a novel semi-synthetic derivative or rapamycin, a naturally product isolated from Streptomyces hydroscopicus. Zotarolimus is prepared by substituting a tetrazole moiety for the hydroxyl group at position 42 of rapamycin. Zotarolimus is an extremely lipophilic molecule which is an advantageous property with regard to delivery of the compound from a drug reservoir layer of an implantable medical device such as, without limitation, a stent. That is, the compound's hydrophobicity and consequent low water solubility permits slow sustained release from a drug reservoir layer which in turn facilitates maintenance of therapeutic drug levels eluting from the drug reservoir layer of the stent over a wide range of delivery times. Further, its lipophilic character favors penetration of cell membranes where it can then inhibit neointimal proliferation of target tissues. Zotarolimus may be administered alone or in conjunction with, that is, before, concurrent with, or after, administration of one or more other therapeutic agent. At present it is preferred to administer zotarolimus alone to treat vascular diseases such as, in particular, restenosis.

The amount of zotarolimus in a drug reservoir layer of this invention is from about 10 μg/cm² to about 1000 μg/cm², preferably about 50 μg/cm² to about 500 μg/cm² and, most preferably at present, about 100 μg/cm².

Therapeutic agents that may be used in conjunction with zotarolimus include, without limitation, antiproliferative agents, anti-inflammatory agents, antineoplastics and/or antimitotics, antiplatelet, anticoagulant, antifibrin, and antithrombin drugs, cytostatic or antiproliferative agents, antibiotics, antiallergic agents and antioxidants.

Suitable antiproliferative agents that can be used in conjunction with zotarolimux include, without limitation, actinomycin D, taxol, docetaxel, paclitaxel, FKBP-12 mediated mTOR inhibitors, perfenidone and prodrugs, co-drugs and combinations thereof.

Suitable anti-inflammatory agents that can be used in conjunction with zotarolimus include, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus and prodrugs, co-drugs and combinations thereof.

Suitable antineoplastics and/or antimitotics that can be used in conjunction with zotarolimus include, without limitation, paclitaxel, docetaxel, methotrexate, azathioprine, vincristine, vinblastine, fluorouracil, doxorubicin hydrochloride, and mitomycin.

Suitable antiplatelet, anticoagulant, antifibrin, and antithrombin drugs that can be used in conjunction with zotarolimus include, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as Angiomax ä, calcium channel blockers (such as nifedipine), colchicine, fish oil (omega 3-fatty acid), histamine antagonists, lovastatin, monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol and combinations thereof.

Suitable cytostatic or antiproliferative agents that can be used in conjunction with zotarolimus include, without limitation, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Other suitable therapeutic agents that can be used in conjunction with zotarolimus include, without limitation, alpha-interferon, genetically engineered epithelial cells, DNA and RNA nucleic acid sequences, antisense molecules and ribozymes.

Further examples of suitable therapeutic agents that can be used in conjunction with zotarolimus include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides; antiviral agents; analgesics; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers, beta-blockers such as pindolol, antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers and natural or genetically engineered lipoproteins.

As used herein, “eluting” as relating zotarolimus or any other therapeutic agent begin released from a drug reservoir layer of this invention refers to the exodus of zotarolimus and/or the other therapeutic agent from the drug reservoir layer into the surrounding environment. The “surrounding environment” ordinarily will constitute the lumen of a vessel or the wall of that lumen which in turn may constitute release directly into cells forming the wall or into the intercellular space.

While any polymer may be used to construct a drug reservoir layer of this invention, preferred polymers are lipophilic to accommodate the highly lipophilic zotarolimus. A presently preferred polymer is poly(vinylidene fluoride-co-hexafluoropropylene) (PVDF). For the purposes of this invention a poly(vinylidene fluoride-co-hexafluoropropylene) having a weight average molecular weight of from about 50,000 to about 500,000 Daltons is present preferred. When poly(vinylidene fluoride-co-hexafluoropropylene) is used as the polymer of the drug reservoir layer, the weight ratio of drug (zotarolimus) to polymer (D:P) is about 1:1 to about 1:10, preferably about 1:3 to about 1:5 and most preferably at present about 1:3.

### EXAMPLES

### Example 1

### Total Recovery of Zotarolimus from a PVDF Drug Reservoir Layer at Various Drug:Polymer (D:P) Ratios

Non-sterile stents with zotarolimus containing PVDF drug reservoir layers were used for this experiment. After the stents had been coated and crimped the stents were balloon expanded into acetonitrile. The stents were sonicated for 30 minutes to allow complete extraction of the drug. The drug content was determined by using high pressure liquid chromatography (HPLC).

As can be seen in Table 1, as the D:P ratio was decreased from 1:3 to 1:4.9 the total drug release decreased from about 22% to about 13% at day 1. That is, at a D:P of 1:3, a total drug release at one day of about 22% and about 40% at day 3. At a D:P of 1:4.9 the total drug release at one day was about 13% and at 3 days about 23%.

TABLE 1

| Drug to | | | |
|---|---|---|---|
| polymer | Percent drug | Release rate at one | Release rate at three |
| ratio (D:P) | recovery (n = 3) | day (%) (n = 3) | days (%) (n = 3) |
| 1:3 | 97.5 ± 0.7 | 22.4 ± 1.1 | 40.8 ± 0.9 |
| 1:4 | 98.2 ± 2.4 | 16.8 ± 1.5 | 28.8 ± 0.7 |
| 1:4.9 | 95.9 ± 0.3 | 13.3 ± 0.8 | 23.0 ± 1.5 |

### Example 2

### Comparison of Total Drug Recovery and Drug Release Rate from Non-Sterile and Ethylene Oxide (EtO) Sterilized Stent

Sterile and non-sterile stents with zotarolimus containing PVDF drug reservoir layers were used for this experiment. After the stents had been coated and crimped the stents used for total drug recovery experiments were either directly balloon expanded into acetonitrile, or first packaged and sterilized and then balloon expanded into acetonitrile. These stents were sonicated for 30 minutes to allow complete extraction of the drug. The drug content was determined by using high pressure liquid chromatography (HPLC).

The stents used to evaluate drug release rate were sterilized and then balloon expanded into porcine serum. The stents were sonicated for 1 day or 3 days and then removed from the porcine serum elution media and rinsed with water. The dry stents were then moved into acetonitrile and sonicated for 30 minutes to allow complete extraction of the remaining drug. The remaining drug content was determined by using high pressure liquid chromatography (HPLC) and the amount released at the various time points was calculated using the experimentally determined total content value. Different stents were used to determine the amount release at 1 and 3 days, respectively.

Table 2 shows the results for drug recovery and drug release from non-sterile stents and stents sterilized by the above procedure.

TABLE 2

| | | Total drug | | |
|---|---|---|---|---|
| | Total drug | recovery | Release | Release |
| | recovery (%), | (%), EtO | rate, one | rate, three |
| Polymer/ | unsterilized | sterilization | day (%) | day (%) |
| drug | (n = 5) | (n = 5) | (n = 5) | (n = 5) |
| PVDF/ | 95.9 ± 0.9 | 95.0 ± 0.5 | 26.27 ± 1.85 | 48.62 ± 3.0 |
| Everolimus | | | | |
| PVDF/ | 95.3 ± 1.6 | 94.9 ± 1.2 | 20.77 ± 1.36 | 43.11 ± 1.3 |
| Zotarolimus | | | | |

What is claimed:

1. An implantable medical device comprising;
a device body;
an optional primer layer disposed over at least a portion of the device body;
a drug reservoir layer disposed over at least a portion of the device body or, if opted for, over the primer layer, wherein
the drug reservoir layer comprises poly(vinylidene fluoride-co-hexafluoropropylene), zotarolimus, optionally one or more additional therapeutic agents; and
an optional topcoat layer.

2. The implantable medical device of claim 1, wherein the weight ratio of zotarolimus to poly(vinylidene fluoride-co-hexafluoropropylene) is about 1:1 to about 1:10.

3. The implantable medical device of claim 2, wherein the weight ratio of zotarolimus to poly(vinylidene fluoride-co-hexafluoropropylene) is about 1:3 to about 1:5.

4. The implantable medical device of claim 3, wherein the weight ratio of zotarolimus to poly(vinylidene fluoride-co-hexafluoropropylene) is about 1:3.

5. The implantable medical device of claim 1, wherein zotarolimus I present in the drug delivery layer at about 10 μg/cm² to about 1000 μg/cm².

6. The implantable medical device of claim 5, wherein zotarolimus is present in the drug delivery layer at about 50 μg/cm² to about 500 μg/cm².

7. The implantable medical device of claim 6, wherein zotarolimus is present in the drug delivery layer at about 100 μg/cm².

8. The implantable medical device of claim 4, wherein approximately 23% of the zotarolimus elutes from the drug reservoir layer into the surrounding environment after about 24 hours.

9. The implantable medical device of claim 4, wherein approximately 40% of the zotarolimus elutes from the drug reservoir layer into the surrounding environment after about 40 hours.

10. The implantable medical device of claim 1, where the device is sterile.

11. The implantable medical device of claim 10, wherein the device is sterilized by exposure to ethylene oxide.

12. The implantable medical device of claim 1, wherein the topcoat layer is not opted for.

13. The implantable medical device of claim 1, wherein the primer layer is not opted for.

14. The implantable medical device of claim 1, wherein neither the topcoat layer nor the primer layer is not opted for.

15. The implantable medical device of claim 1, wherein the device body comprises a stent.

16. The implantable medical device of claim 14 wherein the device body comprises a stent.

17. A method comprising:
identifying a patient afflicted with a vascular disease;
inserting an implantable device percutaneously into a vessel of the patient at a site of the vascular disease, wherein the implantable medical device comprises:
a device body;
an optional primer layer disposed over at least a portion of the device body;
a drug reservoir layer disposed over at least a portion of the device body or, if opted for, over the primer layer, wherein
the drug reservoir layer comprises poly(vinylidene fluoride-co-hexafluoropropylene), zotarolimdus, optionally one or more additional therapeutic agents; and
an optional topcoat layer.

18. The method of claim 17, wherein the topcoat layer is not opted for.

19. The method of claim 18, wherein the primer layer is not opted for.

20. The method of claim 19, wherein no additional therapeutic agent is opted for.

21. The method of claim 20, wherein the device body comprises a stent.

22. The method of claim 20, wherein the vascular disease is selected from the group consisting of atherosclerosis, restenosis, vulnerable plaque and superficial femoral artery sclerosis.
